(19) United States
(12) Patent Application Publication
(10) Pub. No.: US 2008/0280988 A1
(43) Pub. Date: Nov. 13, 2008

(54) Use of (N'-Methyl) benzoylurea compound

(76) Inventors: Norihisa Sakamoto, Sanda-shi (JP); Masato Konobe, Toyonaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 12/081,682

(22) Filed: Apr. 18, 2008

(30) Foreign Application Priority Data
Apr. 19, 2007 (JP) 2007-110172

(51) Int. Cl. A01N 37/22; C07C 237/30; A01P 7/04

(52) U.S. Cl. 514/616; 564/163

(57) ABSTRACT

The present invention provides, as novel use of a certain (N′-methyl)benzoylurea compound in soil treatment for protecting the aerial part of a plant from damage by a pest, a method for protecting the aerial part of a plant from damage by a pest, which comprises a step of applying a (N′-methyl)benzoylurea compound represented by the formula (I):

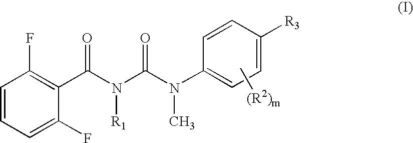

wherein R¹ represents a hydrogen atom, a C1-C6 alkyl group optionally substituted with a halogen atom, or the like, R² represents a halogen atom, or the like, and R³ represents a halogen atom, a C1-C4 alkyl group optionally substituted with a halogen atom, a C1-C4 alkoxy group optionally substituted with a halogen atom, or the like; to soil where the plant is grown.

### BACKGROUND OF THE INVENTION

The present invention relates to novel use of a certain (N′-methyl)benzoylurea compound in soil treatment for protecting the aerial part of a plant from damage by a pest.

It has been found that an N′-methylbenzoylurea compound having a methyl group at the N′-position of benzoylurea can be applied directly to a pest or a food for a pest to control the pest (see JP-A 02-3659 and JP-A 04-26667).

### SUMMARY OF THE INVENTION

An object of the present invention is to provide novel use of a certain (N′-methyl)benzoylurea compound in soil treatment for protecting the aerial part of a plant from damage by a pest.

Inventors of the present invention intensively studied and as a result, completed the present invention. The present invention provides:

[1] use of an (N′-methyl)benzoylurea compound represented by the formula (I):

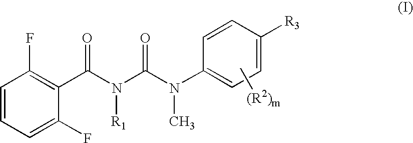

wherein R¹ represents a hydrogen atom, a C1-C6 alkyl group optionally substituted with a halogen atom, a C2-C6 alkenyl group optionally substituted with a halogen atom, a C2-C6 alkynyl group, a C6-C14 aryl group, a C7-C11 aralkyl group, a C2-C6 alkoxyalkyl group, a C7-C14 aryloxyalkyl group, a C3-C6 dialkylaminoalkyl group, a C2-C6 alkylthioalkyl group, a C2-C6 alkylsulfinylalkyl group, a C2-C6 alkylsulfonylalkyl group, a C3-C9 alkoxyalkoxyalkyl group, a C2-C6 alkoxycarbonyl group, a C8-C12 aralkyloxycarbonyl group, a C3-C13 dialkylcarbamoyl group, a C2-C6 alkylcarbonyl group optionally substituted with a halogen atom, a formyl group, a C1-C5 alkylsulfonyl group optionally substituted with a halogen atom, or a C6-C10 arylsulfonyl group,

R² represents a halogen atom, or a C1-C4 alkyl group optionally substituted with a halogen atom, and

R³ represents a halogen atom, a C1-C4 alkyl group optionally substituted with a halogen atom, a C1-C4 alkoxy group optionally substituted with a halogen atom, a C2-C6 alkoxyalkoxy group optionally substituted with a halogen atom, a C2-C4 alkenyloxy group optionally substituted with a halogen atom, or a C2-C4 alkynyloxy group optionally substituted with a halogen atom, and

m represents an integer of 0 to 4; in soil treatment for protecting the aerial part of a plant from damage by a pest;

[2] the use according to the above [1], wherein the pest is a lepidopteran or a thrips;
[3] a composition for soil treatment for protecting the aerial part of a plant from damage by a pest, which comprises an (N′-methyl)benzoylurea compound represented by the above formula (I);
[4] a method for protecting the aerial part of a plant from damage by a pest, which comprises a step of applying the composition according to the above [3] to soil where the plant is grown;
[5] the method according to the above [4], wherein the plant is a seedling;
[6] the method according to the above [4] or [5], wherein the pest is a lepidopteran or a thrips; and
[7] the method according to the above [4] or [5], wherein application of the composition is carried out by soil drenching.

According to the present invention, the aerial part of a plant can be protected from damage by a pest by applying the composition of the present invention comprising an (N′-methyl)benzoylurea compound of the formula (I) (hereinafter, referred to as the present compound) to soil where the plant is grown.

### DETAILED DESCRIPTION OF THE PREFERRED EMBODIMENTS

Examples of the “C1-C6 alkyl group optionally substituted with a halogen atom” represented by R¹, as used herein, include a methyl group, a chloromethyl group, a difluoromethyl group, a trichloromethyl group, an ethyl group, a 2-bromoethyl group, a 2,2,2-trifluoroethyl group, a propyl group, a 3,3,3-trifluoropropyl group, an isopropyl group, a butyl group, an isobutyl group, a sec-butyl group, a tert-butyl group, a 4,4,4-trifluorobutyl group, a pentyl group, an isopentyl group, a neopentyl group, a 5,5,5-trifluoropentyl group, a hexyl group and a 6,6,6-trifluorohexyl group.

Examples of the “C2-C6 alkenyl group optionally substituted with a halogen atom” represented by R¹, as used herein, include a vinyl group, a 1-propenyl group, a 2-propenyl group, an isopropenyl group, a 2-butenyl group, an isobutenyl group and a 3,3-dichloro-2-propenyl group.

Examples of the “C2-C6 alkynyl group” represented by R¹, as used herein, include an ethynyl group, a 2-propynyl group and a 1-propynyl group.

Examples of the “C6-C14 aryl group” represented by R¹, as used herein, include a phenyl group, a 1-naphthyl group, a 2-naphthyl group and a biphenylyl group.

Examples of the “C7-C11 aralkyl group” represented by R¹, as used herein, include a benzyl group and a phenethyl group.

Examples of “C2-C6 alkoxyalkyl group” represented by R¹, as used herein, include a methoxymethyl group, an ethoxymethyl group, a 1-propoxymethyl group, a 2-methoxyethyl group, a 2-ethoxyethyl group, a 3-methoxypropyl group and a 3-ethoxypropyl group.

Examples of the “C7-C14 aryloxyalkyl group” represented by R¹, as used herein, include a phenoxymethyl group and a 2-phenoxyethyl group.

Examples of the “C3-C6 dialkylaminoalkyl group” represented by R¹, as used herein, include a dimethylaminomethyl group, a 2-(dimethylamino)ethyl group, a diethylaminomethyl group and a 2-(diethylamino)ethyl group.

Examples of the “C2-C6 alkylthioalkyl group” represented by R¹, as used herein, include a methylthiomethyl group, an ethylthiomethyl group, a 2-(methylthio)ethyl group and a 2-(ethylthio)ethyl group.

Examples of the “C2-C6 alkylsulfinylalkyl group” represented by R¹, as used herein, include a methylsulfinylmethyl group, an ethylsulfinylmethyl group, a 2-(methylsulfinyl)ethyl group and a 2-(ethylsulfinyl)ethyl group.

Examples of the “C2-C6 alkylsulfonylalkyl group” represented by R¹, as used herein, include a methylsulfonylmethyl group, an ethylsulfonylmethyl group, a 2-(methylsulfonyl)ethyl group and a 2-(ethylsulfonyl)ethyl group.

Examples of the “C3-C9 alkoxyalkoxyalkyl group)”represented by R¹, as used herein, include a (2-methoxyethoxy)methyl group.

Examples of the “C2-C6 alkoxycarbonyl group” represented by R¹, as used herein, include a methoxycarbonyl group, an ethoxycarbonyl group, a n-propoxycarbonyl group, an isopropoxycarbonyl group, a n-butoxycarbonyl group and a tert-butoxycarbonyl group.

Examples of the “C8-C12 aralkyloxycarbonyl group” represented by R¹, as used herein, include a benzyloxycarbonyl group.

Examples of the “C3-C13 dialkylcarbamoyl group” represented by R¹, as used herein, include a dimethylcarbamoyl group and a diethylcarbamoyl group.

Examples of the “C2-C6 alkylcarbonyl group optionally substituted with a halogen atom” represented by R¹, as used herein, include an acetyl group, a propionyl group, a trifluoroacetyl group and a chloroacetyl group.

Examples of the “C1-C5 alkylsulfonyl group optionally substituted with a halogen atom” represented by R¹, as used herein, include a methanesulfonyl group, an ethanesulfonyl group and a trifluoromethanesulfonyl group.

Examples of the “C6-C10 arylsulfonyl group” represented by R¹, as used herein, include a benzenesulfonyl group and a toluenesulfonyl group.

Examples of the “halogen atom” represented by R², as used herein, include a fluorine atom, a chlorine atom, a bromine atom and an iodine atom.

Examples of the “C1-C4 alkyl group optionally substituted with a halogen atom” represented by R², as used herein, include a methyl group, a chloromethyl group, a difluoromethyl group, a trichloromethyl group, a trifluoromethyl group, an ethyl group, a 2-bromoethyl group, a 2,2,2-trifluoroethyl group, a pentafluoroethyl group, a propyl group, a 3,3,3-trifluoropropyl group, an isopropyl group, a butyl group, an isobutyl group, a sec-butyl group, a tert-butyl group and a 4,4,4-trifluorobutyl group.

Examples of the “halogen atom” represented by R³, as used herein, include a fluorine atom, a chlorine atom, a bromine atom and an iodine atom.

Examples of the “C1-C4 alkyl group optionally substituted with a halogen atom” represented by R³, as used herein, include a methyl group, a chloromethyl group, a difluoromethyl group, a trichloromethyl group, a trifluoromethyl group, an ethyl group, a 2-bromoethyl group, a 2,2,2-trifluoroethyl group, a pentafluoroethyl group, a propyl group, a 3,3,3-trifluoropropyl group, an isopropyl group, a butyl group, an isobutyl group, a sec-butyl group, a tert-butyl group and a 4,4,4-trifluorobutyl group.

Examples of the “C1-C4 alkoxy group optionally substituted with a halogen atom” represented by R³, as used herein, include a methoxy group, an ethoxy group, a 1-propyloxy group, an isopropoxy group, a tert-butoxy group, a difluoromethoxy group, a trifluoromethoxy group, a 2,2,2-trifluoroethoxy group, a 1,1,2,2,2-pentafluoroethoxy group, a 1,1,2,2-tetrafluoroethoxy group, a 1,1,2,2,2,3,3,3-heptafluoro-1-propoxy group and a 1,1,2,3,3,3-hexafluoro-1-propoxy group.

Examples of the “C2-C6 alkoxyalkoxy group optionally substituted with a halogen atom” represented by R³, as used herein, include a 2-trifluoromethoxy-1,1,2-trifluoroethoxy group.

Examples of the “C2-C4 alkenyloxy group optionally substituted with a halogen atom” represented by R³, as used herein, include a 2-propenyloxy group and a 3,3-dichloro-2-propenyloxy group.

Examples of the “C2-C4 alkynyloxy group optionally substituted with a halogen atom” represented by R³, as used herein, include a 2-propynyloxy group.

Then, processes for synthesizing of the present compound are shown.

The present compound can be produced, for example, by the following Synthesis Processes 1 to 4.

### (Synthesis Process 1)

Among the present compounds, a compound represented by the formula (I-1):

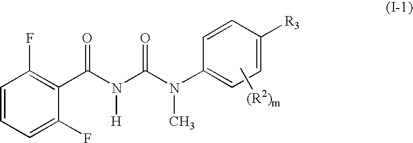

wherein R², R³ and m are as defined above, can be produced by reacting a compound represented by the formula (II):

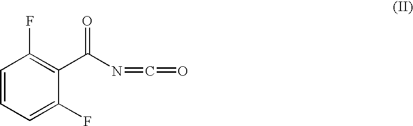

wherein X and Y are as defined above, with a compound represented by the formula (III):

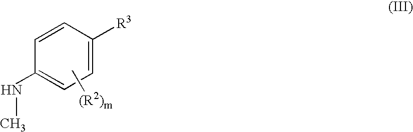

wherein R², R³ and m are as defined above.

The reaction is usually performed in a solvent.

Examples of a solvent used in the reaction include ketones such as acetone and methyl ethyl ketone, aromatic hydrocarbons such as benzene, toluene and xylene, aliphatic hydrocarbons such as hexane and heptane, ethers such as diethyl ether, tetrahydrofuran, 1,4-dioxane, 1,2-dimethoxyethane and 1,2-diethoxyethane, halogenated hydrocarbons such as chloroform, chlorobenzene and dichlorobenzene, nitrites such as acetonitrile, aprotic polar solvents such as N,N-dimethylformamide, N,N-dimethylacetamide, 1-methyl-2-pyrrolidone, 1,3-dimethylimidazolidinone and dimethyl sulfoxide, and their mixtures.

In the reaction, usually 0.5 to 2 mol of the compound represented by the formula (III) is used per 1 mol of the compound represented by the formula (II).

The reaction temperature is usually in a range of −78 to 150° C. The reaction time is usually in a range of 0.1 to 100 hours.

After completion of the reaction, the compound represented by the formula (I-1) can be isolated by posttreatment, for example, by pouring a reaction mixture into water, extracting the mixture with an organic solvent, and then drying and concentrating an organic layer. The isolated compound represented by the formula (I-1) can be further purified by recrystallization, column chromatography or the like.

### (Synthesis Process 2)

Among the present compounds, a compound represented by the formula (I-2):

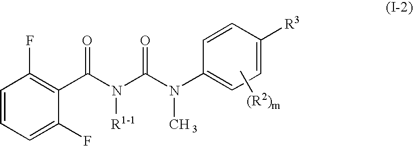

wherein R², R³ and m are as defined above, and R¹⁻¹ represents a C1-C6 alkyl group optionally substituted with a halogen atom, a C2-C6 alkenyl group optionally substituted with a halogen atom, a C2-C6 alkynyl group, a C6-C14 aryl group, a C7-C11 aralkyl group, a C2-C6 alkoxyalkyl group, a C7-C14 aryloxyalkyl group, a C3-C6 N,N-di(alkyl)aminoalkyl group, a C2-C6 alkylthioalkyl group, a C2-C6 alkylsulfinylalkyl group, a C2-C6 alkylsulfonylalkyl group or a C3-C9 alkoxyalkoxyalkyl group, can be produced by reacting a compound represented by the formula (IV):

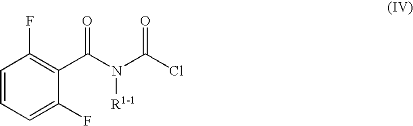

wherein X, Y and R¹⁻¹ are as defined above, with the compound represented by the formula (III).

The reaction is performed in a conventional solvent in the presence of a base.

Examples of a solvent used in the reaction include ketones such as acetone and methyl ethyl ketone, aromatic hydrocarbons such as benzene, toluene and xylene, aliphatic hydrocarbons such as hexane and heptane, ethers such as diethyl ether, tetrahydrofuran, 1,4-dioxane, 1,2-dimethoxyethane and 1,2-diethoxyethane, halogenated hydrocarbons such as chloroform, chlorobenzene and dichlorobenzene, nitrites such as acetonitrile, aprotic polar solvents such as N,N-dimethylformamide, N,N-dimethylacetamide, 1-methyl-2-pyrrolidone, 1,3-dimethylimidazolidinone and dimethyl sulfoxide, and their mixtures.

Examples of a base used in the reaction include hydroxides of alkali metals or alkaline earth metals such as sodium hydroxide, potassium hydroxide and calcium hydroxide, hydrides of alkali metals or alkaline earth metals such as sodium hydride, potassium hydride and calcium hydride, carbonates of alkali metals or alkaline earth metals such as sodium carbonate and potassium carbonate, alcoholates of alkali metals such as sodium ethylate and sodium methylate, organic lithium such as n-butyllithium and lithium diisopropylamide, and organic bases such as triethylamine, pyridine and 1,8-diazabicyclo[5,4,0]undecene (hereinafter, abbreviated as DBU).

When reagents are in liquid form under a reaction condition, a solvent amount of each reagent may be used in the reaction. Usually, 1 to 4 mol of the compound represented by the formula (III) and 1 to 4 mol of the base are used per 1 mol of the compound represented by the formula (IV).

The reaction temperature is usually in a range of −78 to 150° C. The reaction time is usually in a range of 0.1 to 200 hours.

After completion of the reaction, the compound represented by the formula (I-2) can be isolated by posttreatment, for example, by pouring a reaction mixture into water, extracting the mixture with an organic solvent, and then drying and concentrating an organic layer. The isolated compound represented by the formula (I-2) can be further purified by recrystallization, column chromatography or the like.

### (Synthesis Process 3)

Among the present compounds, a compound represented by the formula (I-3):

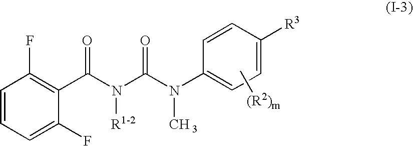

wherein R², R³ and m are as defined above, and R¹⁻² represents a C1-C6 alkyl group optionally substituted with a halogen atom, a C2-C6 alkenyl group optionally substituted with a halogen atom, a C2-C6 alkynyl group, a C7-C11 aralkyl group, a C2-C6 alkoxyalkyl group, a C7-C14 aryloxyalkyl group, a C3-C6 dialkylaminoalkyl group, a C2-C6 alkylthioalkyl group, a C2-C6 alkylsulfinylalkyl group, a C2-C6 alkylsulfonylalkyl group, a C3-C9 alkoxyalkoxyalkyl group, a C2-C6 alkoxycarbonyl group, a C8-C12 aralkyloxycarbonyl group, a C3-C13 dialkylcarbamoyl group, a C2-C6 alkylcarbonyl group optionally substituted with a halogen atom, a formyl group, a C1-C5 alkylsulfonyl group optionally substituted with a halogen atom, or a C6-C10 arylsulfonyl group, can be produced by reacting the compound represented by the formula (I-1) with a compound represented by the formula (V):

L¹-R¹⁻² (V)

wherein R¹⁻² is as defined above, and L¹ represents a halogen atom (e.g. a chlorine atom or a bromine atom), a methanesulfonyloxy group, a benzenesulfonyloxy group, a toluenesulfonyloxy group, a methoxysulfonyloxy group or an ethoxysulfonyloxy group, in the presence of a base.

The reaction is usually performed in a conventional solvent in the presence of a base.

Examples of a solvent used in the reaction include ketones such as acetone and methyl ethyl ketone, aromatic hydrocarbons such as benzene, toluene and xylene, aliphatic hydrocarbons such as hexane and heptane, ethers such as diethyl ether, tetrahydrofuran, 1,4-dioxane, 1,2-dimethoxyethane and 1,2-diethoxyethane, halogenated hydrocarbons such as chloroform, chlorobenzene and dichlorobenzene, nitrites such as acetonitrile, aprotic polar solvents such as N,N-dimethylformamide, N,N-dimethylacetamide, 1-methyl-2-pyrrolidone, 1,3-dimethylimidazolidone and dimethyl sulfoxide, and their mixtures.

Examples of a base used in the reaction include hydroxides of alkali metals or alkaline earth metals such as sodium hydroxide, potassium hydroxide and calcium hydroxide, hydrides of alkali metals or alkaline earth metals such as sodium hydride, potassium hydride and calcium hydride, carbonates of alkali metals or alkaline earth metals such as sodium carbonate and potassium carbonate, alcoholates of alkali metals such as sodium ethylate and sodium methylate, organic lithium such as n-butyllithium and lithium diisopropylamide, and organic bases such as triethylamine, pyridine and DBU.

When reagents are in liquid form under a reaction condition, a solvent amount of each reagent may be used in the reaction. Usually, 1 to 4 mol of the compound represented by the formula (V) and 1 to 4 mol of the base are used per 1 mol of the compound represented by the formula (I-1).

The reaction temperature is usually in a range of −78 to 150° C. The reaction time is usually in a range of 0.1 to 100 hours.

After completion of the reaction, the compound represented by the formula (I-3) can be isolated by posttreatment, for example, by pouring a reaction mixture into water, extracting the mixture with an organic solvent, and then drying and concentrating an organic layer. The isolated compound represented by the formula (I-3) can be further purified by recrystallization, column chromatography or the like.

### (Production Process 4)

Among the present compounds, a compound represented by the formula (I-2):

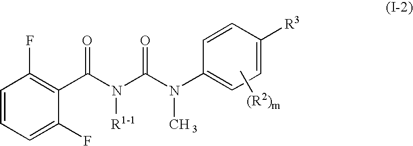

wherein R², R³, R¹⁻¹ and m are as defined above, can be produced by reacting a compound represented by the formula (VI):

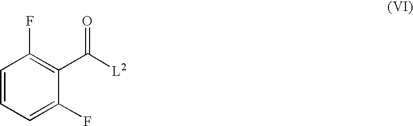

wherein X and Y are as defined above and L² represents a halogen atom (e.g. a chlorine atom, a bromine atom or a iodine atom), with a compound represented by the formula (VII):

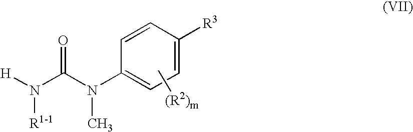

wherein R¹⁻¹, R², R³ and m are as defined above.

The reaction is performed in a conventional solvent in the presence of a base.

Examples of a solvent used in the reaction include ketones such as acetone and methyl ethyl ketone, aromatic hydrocarbons such as benzene, toluene and xylene, aliphatic hydrocarbons such as hexane and heptane, ethers such as diethyl ether, tetrahydrofuran, 1,4-dioxane, 1,2-dimethoxyethane and 1,2-diethoxyethane, halogenated hydrocarbons such as chloroform, chlorobenzene and dichlorobenzene, nitrites such as acetonitrile, aprotic polar solvents such as N,N-dimethylformamide, N,N-dimethylacetamide, 1-methyl-2-pyrrolidone, 1,3-dimethylimidazolidone and dimethyl sulfoxide, and their mixtures.

Examples of a base used in the reaction include hydroxides of alkali metals or alkaline earth metals such as sodium hydroxide, potassium hydroxide and calcium hydroxide, hydrides of alkali metals or alkaline earth metals such as sodium hydride, potassium hydride and calcium hydride, carbonates of alkali metals or alkaline earth metals such as sodium carbonate and potassium carbonate, alcoholates of alkali metals such as sodium ethylate and sodium methylate, organic lithium such as n-butyllithium and lithium diisopropylamide, and organic bases such as triethylamine, diisopropylethylamine, pyridine and DBU.

When reagents are in liquid form under a reaction condition, a solvent amount of each reagent may be used in the reaction. Usually, 1 to 4 mol of the compound represented by the formula (VI) and 1 to 4 mol of the base are used per 1 mol of the compound represented by the formula (VII).

The reaction temperature is usually in a range of −78 to 150° C. The reaction time is usually in a range of 0.1 to 200 hours.

After completion of the reaction, the compound represented by the formula (I-2) can be isolated by posttreatment, for example, by pouring a reaction mixture into water, extracting the mixture with an organic solvent, and then drying and concentrating an organic layer. The isolated compound represented by the formula (I-2) can be further purified by recrystallization, column chromatography or the like.

Then, Reference Synthesis Processes of intermediates for synthesis of the present compound are shown.

### (Reference Synthesis Process 1)

The compound represented by the formula (IV) can be produced by reacting a compound represented by the formula (VIII):

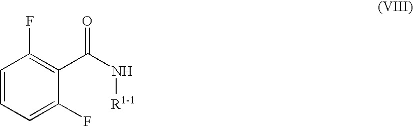

wherein X, Y and R¹⁻¹ are as defined above, with a trialkylchlorosilane compound and a chlorocarbonylating agent.

The reaction is performed in a conventional solvent in the presence of a base.

Examples of a solvent used in the reaction include ketones such as acetone and methyl ethyl ketone, aromatic hydrocarbons such as benzene, toluene and xylene, aliphatic hydrocarbons such as hexane and heptane, ethers such as diethyl ether, tetrahydrofuran, 1,4-dioxane, 1,2-dimethoxyethane and 1,2-diethoxyethane, halogenated hydrocarbons such as chloroform, chlorobenzene and dichlorobenzene, nitrites such as acetonitrile, aprotic polar solvents such as N,N-dimethylformamide, N,N-dimethylacetamide, 1-methyl-2-pyrrolidone, 1,3-dimethylimidazolidone and dimethyl sulfoxide, and their mixtures.

Examples of a base used in the reaction include hydroxides of alkali metals or alkaline earth metals such as sodium hydroxide, potassium hydroxide and calcium hydroxide, hydrides of alkali metals or alkaline earth metals such as sodium hydride, potassium hydride and calcium hydride, carbonates of alkali metals or alkaline earth metals such as sodium carbonate and potassium carbonate, alcoholates of alkali metals such as sodium ethylate and sodium methylate, organic lithium such as n-butyllithium and lithium diisopropylamide, and organic bases such as triethylamine, pyridine and DBU.

Examples of a trialkylchlorosilane compound used in the reaction include trimethylchlorosilane and triethylchlorosilane.

Examples of a chlorocarbonylating agent used in the reaction include phosgene, trichloromethyl chloroformate and bis(trichloromethyl) carbonate.

In the reaction, usually 1 to 4 mol of the trialkylchlorosilane compound, 1 to 4 mol of the chlorocarbonylating agent and 1 to 4 mol of the base are used per 1 mol of the compound represented by the formula (VIII).

The reaction temperature is usually in a range of −78 to 150° C. The reaction time is usually in a range of 0.1 to 200 hours.

After completion of the reaction, the compound represented by the formula (IV) can be isolated by posttreatment, for example, by concentrating a reaction mixture as it is. The isolated compound represented by the formula (IV) can be used in the next step without purification.

### (Reference Production Process 2)

The compound represented by the formula (VII) can be produced by reacting a compound represented by the formula (IX):

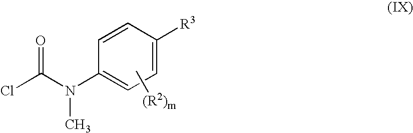

wherein R², R³ and m are as defined above, with a compound represented by the formula (X):

H₂N—R¹⁻¹ (X)

wherein R¹⁻¹ is as defined above.

The reaction is performed in a conventional solvent in the presence of a base.

Examples of a solvent used in the reaction include ketones such as acetone and methyl ethyl ketone, aromatic hydrocarbons such as benzene, toluene and xylene, aliphatic hydrocarbons such as hexane and heptane, ethers such as diethyl ether, tetrahydrofuran, 1,4-dioxane, 1,2-dimethoxyethane and 1,2-diethoxyethane, halogenated hydrocarbons such as chloroform, chlorobenzene and dichlorobenzene, nitrites such as acetonitrile, aprotic polar solvents such as N,N-dimethylformamide, N,N-dimethylacetamide, 1-methyl-2-pyrrolidone, 1,3-dimethylimidazolidone and dimethyl sulfoxide, and their mixtures.

Examples of a base used in the reaction include hydroxides of alkali metals or alkaline earth metals such a sodium hydroxide, potassium hydroxide and calcium hydroxide, hydrides of alkali metals or alkaline earth metals such as sodium hydride, potassium hydride and calcium hydride, carbonates of alkali metals or alkaline earth metals such as sodium carbonate and potassium carbonate, alcoholates of alkali metals such as sodium ethylate and sodium methylate, organic lithium such as n-butyllithium and lithium diisopropylamine, and organic bases such as triethylamine and DBU. Alternatively, an excessive amount of a compound represented by the formula (X) can be used as the base.

In the reaction, usually 1 to 6 mol of the compound represented by the formula (X) and 1 to 6 mol of the base are used per 1 mol of the compound represented by the formula (IX).

The reaction temperature is usually in a range of −78 to 150° C. The reaction time is usually in a range of 0.1 to 200 hours.

After completion of the reaction, the compound represented by the formula (VII) can be isolated by posttreatment, for example, by pouring a reaction mixture into water, extracting the mixture with an organic solvent, and then drying and concentrating an organic layer. The isolated compound represented by the formula (VII) can be further purified by recrystallization, column chromatography or the like.

### (Reference Production Process 3)

The compound represented by the formula (IX) can be produced by reacting a compound represented by the formula (III) with a chlorocarbonylating agent.

The reaction is performed in a conventional solvent in the presence of a base.

Examples of a solvent used in the reaction include ketones such as acetone and methyl ethyl ketone, aromatic hydrocarbons such as benzene, toluene and xylene, aliphatic hydrocarbons such as hexane and heptane, ethers such as diethyl ether, tetrahydrofuran, 1,4-dioxane, 1,2-dimethoxyethane and 1,2-diethoxyethane, halogenated hydrocarbons such as chloroform, chlorobenzene and dichlorobenzene, nitrites such as acetonitrile, aprotic polar solvents such as N,N-dimethylformamide, N,N-dimethylacetamide, 1-methyl-2-pyrrolidone, 1,3-dimethylimidazolidone and dimethyl sulfoxide, and their mixtures.

Examples of a base used in the reaction include hydroxides of alkali metals or alkaline earth metals such as sodium hydroxide, potassium hydroxide and calcium hydroxide, hydrides of alkali metals or alkaline earth metals such as sodium hydride, potassium hydride and calcium hydride, carbonates of alkali metals or alkaline earth metals such as sodium carbonate and potassium carbonate, alcoholates of alkali metals such as sodium ethylate and sodium methylate, organic lithium such as n-butyllithium and lithium diisopropylamide, and organic bases such as triethylamine, pyridine and DBU.

Examples of a chlorocarbonylating agent used in the reaction include phosgene, trichloromethyl chloroformate, and bis(trichloromethyl) carbonate.

In the reaction, usually 1 to 4 mol of the chlorocarbonylating agent and 1 to 4 mol of the base are used per 1 mol of the compound represented by the formula (III).

The reaction temperature is usually in a range of −78 to 150° C. The reaction time is usually in a range of 0.1 to 200 hours.

After completion of the reaction, the compound represented by the formula (IX) can be isolated by posttreatment, for example, by pouring a reaction mixture into water, extracting the mixture with an organic solvent, and then drying and concentrating an organic layer. The isolated compound represented by the formula (IX) can be further purified by recrystallization, column chromatography or the like. Alternatively, after completion of the reaction, the compound represented by the formula (IX) can be also isolated by posttreatment such as concentration of a reaction mixture as it is.

Compounds produced by the above-described synthesis processes can be further subjected to a per se known method, such as alkylation, alkynylation, alkynylation, acylation, amination, sulfidization, sulfonylation, sulfonation, oxidation, reduction, halogenation or nitration to convert a substituent into another desired substituent.

Specific examples of the present compound include:

an (N′-methyl)benzoylurea compound represented by the formula (I) wherein R¹ is a C1-C6 alkyl group optionally substituted with a halogen atom, a C2-C6 alkenyl group optionally substituted with a halogen atom, a C2-C6 alkynyl group, a C6-C14 aryl salt, a C7-C11 aralkyl group or a C2-C6 alkoxyalkyl group;

an (N′-methyl)benzoylurea compound represented by the formula (I) wherein R¹ is a hydrogen atom, a C1-C6 alkyl group or a C2-C6 alkoxyalkyl group;

an (N′-methyl)benzoylurea compound represented by the formula (I) wherein R¹ is a hydrogen atom, a methyl group or a methoxymethyl group;

an (N′-methyl)benzoylurea compound represented by the formula (I) wherein m is 0 or 1;

an (N′-methyl)benzoylurea compound represented by the formula (I) wherein m is 1 and R² is a halogen atom;

an (N′-methyl)benzoylurea compound represented by the formula (I) wherein R³ is a halogen atom, a C1-C4 alkoxy group optionally substituted with a halogen atom or a C1-C4 alkyl group optionally substituted with a halogen atom;

an (N′-methyl)benzoylurea compound represented by the formula (I) wherein R³ is a halogen atom, a trifluoromethoxy group, a 1,1,2,2-tetrafluoroethoxy group or a trifluoromethyl group;

an (N′-methyl)benzoylurea compound represented by the formula (I) wherein R¹ is a hydrogen atom, a C1-C6 alkyl group optionally substituted with a halogen atom, a C2-C6 alkenyl group optionally substituted with a halogen atom, a C2-C6 alkynyl group, a C6-C14 aryl group, a C7-C11 aralkyl group or a C2-C6 alkoxyalkyl group, R²is a halogen atom, and R³ is a halogen atom, a C1-C4 alkoxy group optionally substituted with a halogen atom or a C1-C4 alkyl group optionally substituted with a halogen atom; and

an (N′-methyl)benzoylurea compound represented by the formula (I) wherein R¹ is a hydrogen atom, a C1-C6 alkyl group or a C2-C6 alkoxyalkyl group, R² is a halogen atom, and R³ is a halogen atom, a trifluoromethoxy group, a 1,1,2,2-tetrafluoroethoxy group or a trifluoromethyl group.

Preferred examples of the present compound include:
3-(2,6-difluorobenzoyl)-1-[2-fluoro-4-(1,1,2,2-tetrafluoroethoxy)phenyl]-1-methylurea,
3-(2,6-difluorobenzoyl)-1-(4-trifluoromethoxyphenyl)-1-methylurea,
3-(2,6-difluorobenzoyl)-1-(4-chlorophenyl)-1-methylurea,
3-(2,6-difluorobenzoyl)-1-(4-chlorophenyl)-1,3-dimethylurea,
3-(2,6-difluorobenzoyl)-1-(3,5-dichloro-2,4-difluorophenyl)-1,3-dimethylurea,
3-(2,6-difluorobenzoyl)-1-[2-fluoro-4-(1,1,2,2-tetrafluoroethoxy)phenyl]-1,3-dimethylurea,
3-(2,6-difluorobenzoyl)-1-[2-fluoro-4-(trifluoromethyl)phenyl]-1,3-dimethylurea,
3-(2,6-difluorobenzoyl)-1-[2-fluoro-4-(trifluoromethyl)phenyl]-1-methylurea, and
3-(2,6-difluorobenzoyl)-1-[4-(1,1,2,2-tetrafluoroethoxy)phenyl]-1-methylurea.

The method for protecting the aerial part of a plant from damage by a pest of the present invention (hereinafter, referred to as the method of the present invention) comprises a step of applying a composition comprising the present compound to soil where the plant is grown. Although the composition to be applied to soil where a plant is grown (hereinafter, referred to as the composition for soil treatment of the present invention) may be the present compound itself, the composition usually comprises the present compound or a salt thereof in combination with a carrier such as a solid carrier or a liquid carrier and, if necessary, an emulsifying agent, a suspending agent, a spreading agent, a penetrating agent, a wetting agent, a mucilage, a stabilizer or the like. The composition for soil treatment of the present invention may take a liquid form including water dilutions of an emulsifiable concentrate, a liquid formulation, a microemulsion, an emulsion, a flowable formulation, an oil formulation and the like; a solid form including a wettable powder, a water soluble powder, a sol, a dust formulation, a pellet, a tablet and a film formulation; or a capsulated form prepared by encapsulation or overcoating of a formulation containing the present compound.

Examples of the solid carrier include vegetable powder (soybean powder, tobacco powder, wheat powder, wood powder, etc.), mineral powder (clay such as kaolin, bentonite and acid clay, talc such as talcum powder and agalmatolite, silica such as diatomaceous earth and mica powder, etc.), alumina, sulfur powder, activated carbon, calcium carbonate, ammonium sulfate, sodium hydrogen carbonate, lactose, urea, and the like. When the composition for soil treatment of the present invention comprises a solid carrier, the solid carrier may be a mixture of one or more of the aforementioned solid carriers in an appropriate ratio.

Examples of the liquid carrier include water, alcohols (methyl alcohol, ethyl alcohol, n-propyl alcohol, isopropyl alcohol, ethylene glycol, etc.), ketones (acetone, methyl ethyl ketone, methyl isobutyl ketone, cyclohexanone, etc.), ethers (tetrahydrofuran, ethylene glycol monomethyl ether, diethylene glycol monomethyl ether, propylene glycol monomethyl ether, etc.), aliphatic hydrocarbons (kerosene, fuel oil, machine oil, etc.), aromatic hydrocarbons (toluene, xylene, solvent naphtha, methylnaphthalene, etc.), halogenated hydrocarbons (dichloromethane, chloroform, carbon tetrachloride, etc.), acid amides (N,N-dimethylformamide, N,N-dimethylacetamide, N-methylpyrrolidone, etc.), esters (ethyl acetate, butyl acetate, fatty acid glycerin ester, γ-butyrolactone, etc.), and nitrites (acetonitrile, propionitrile, etc.). When the composition for soil treatment of the present invention comprises a liquid carrier, the liquid carrier may be a mixture of one or more of the aforementioned liquid carriers in an appropriate ratio.

An example of the emulsifying agent, the suspending agent, the spreading agent, the penetrating agent, the wetting agent, the mucilage, the stabilizer or the like which the composition for soil treatment of the present invention may contain as necessary is a surfactant. Examples of the surfactant include nonionic and anionic surfactants such as soaps, polyoxyethylene alkylaryl ethers [e.g. NOIGEN (trade name), EA 142 (trade name); manufactured by Dai-ichi Kogyo Seiyaku Co., Ltd., NONAL (trade name); manufactured by Kao Corporation], alkyl sulfate salts [e.g. EMAL 10 (trade name), EMAL 40 (trade name); manufactured by Kao Corporation], alkylbenzenesulfonate salts [e.g. NEOGEN (trade name), NEOGEN T (trade name); manufactured by Daiichi Kogyo Seiyaku Co., Ltd., NEOPELEX (trade name); manufactured by Kao Corporation], polyethylene glycol ethers [e.g. NONIPOL 85 (trade name), NONIPOL 100 (trade name), NONIPOL 160 (trade name); manufactured by Sanyo Chemical Industries], polyhydric alcohol esters [e.g. TWEEN 20 (trade name), TWEEN 80 (trade name); manufactured by Kao Corporation], alkylsulfosuccinate salts [e.g. SANMORIN OT20 (trade name); manufactured by Sanyo Chemical Industries, NEWKALGEN EX70 (trade name); manufactured by Takemoto Oil & Fat Co., Ltd.], alkylnaphthalenesulfonate salts, and alkenylsulfonate salts [e.g. SOLPOL 5115 (trade name); manufactured by Toho Kagaku Co., Ltd.].

The step of applying the composition for soil treatment of the present invention to soil where a plant is grown can be carried out by

1) planting hole application (planting hole spraying, planting hole soil incorporation),

2) plant foot application (plant foot spraying, plant foot soil incorporation, plant foot drenching, plant foot application at a later seeding raising stage),

3) planting furrow application (planting furrow spraying, planting furrow soil incorporation),

4) planting row application (planting row spraying, planting row soil incorporation, planting row spraying at a growing stage),

5) planting row application at sowing (planting row spraying at sowing, planting row soil incorporation at sowing),

6) broadcast application (overall soil surface spraying, overall soil incorporation),

7) drench application (soil drenching, plant foot drenching, chemical drip irrigation, chemigation),

8) nursery box application (nursery box surface spraying, nursery box soil drenching, nursery box soil incorporation),

9) nursery bed application (nursery bed surface spraying, nursery bed drenching, lowland nursery bed surface spraying),

10) bed soil application (bed soil incorporation, presowing bed soil incorporation, bed soil surface spraying after sowing and soil covering),

11) other soil application (paste fertilizer incorporation), or the like, using the composition of the present invention, for example, in the form of a water dilution of an emulsifiable concentrate, a liquid formulation, a microemulsion, an emulsion, a flowable formulation, an oil formulation, a wettable powder, a water soluble powder, a microcapsule or a sol, or in the form of a granule, a tablet or a film formulation. Two or more of the aforementioned application methods may be combined.

Preferably, the step of applying the composition for soil treatment of the present invention to soil where a plant is grown can be carried out by drenching application, more preferably soil drenching.

The application amount of the composition for soil treatment of the present invention in the aforementioned application step may be appropriately changed depending on an application timing, an application place, a formulation form, a subject pest to be controlled and the like, and it is usually 0.3 to 3,000 g, preferably 50 to 3,000 g of the present compound per 1 hectare of soil where a plant is grown.

The composition for soil treatment of the present invention may contain additional active ingredients in addition to the present compound. Examples of the additional active ingredient include insecticides (e.g. pyrethroid insecticides, organophosphate insecticides, carbamate insecticides, neonicotinoid insecticides, natural insecticides), acaricides, machine oils, nematicides, herbicides, plant hormones, plant growth regulating substances, fungicides (e.g. copper fungicides, organic chlorine fungicides, organic sulfur fungicides, phenol fungicides), synergists, attractants, repellants, agents for alleviating chemical injuries, colorants, fertilizers and the like.

Examples of the insecticide include

(1) Organophosphate Insecticides:

aluminum phosphide, butathiofos, cadusafos, chlorethoxyfos, chlorfenvinphos, chlorpyrifos, chlorpyrifos-methyl, cyanophos (CYAP), diazinon, DCIP (dichlorodiisopropyl ether), dichlofenthion (ECP), dichlorvos (DDVP), dimethoate, dimethylvinphos, disulfoton, EPN, ethion, ethoprophos, etrimfos, fenthion (MPP), fenitrothion (MEP), fosthiazate, formothion, hydrogen phosphide, isofenphos, isoxathion, malathion, mesulfenfos, methidathion (DMTP), monocrotophos, naled (BRP), oxydeprofos (ESP), parathion, phorate, phosalone, phosmet (PMP), pirimiphos-methyl, pyridafenthion, quinalphos, phenthoate (PAP), profenofos, propaphos, prothiofos, pyraclorfos, salithion, sulprofos, tebupirimfos, temephos, tetrachlorvinphos, terbufos, thiometon, trichlorphon (DEP), vamidothion, and the like;

(2) Carbamate Insecticides:

alanycarb, aldicarb, bendiocarb, benfuracarb, BPMC, carbaryl, carbofuran, carbosulfan, chloethocarb, ethiofencarb, fenobucarb, fenothiocarb, fenoxycarb, furathiocarb, isoprocarb (MIPC), metalcarb, methomyl, methiocarb, NAC, oxamyl, pirimicarb, propoxur (PHC), XMC, thiodicarb, xylylcarb, and the like;

(3) Synthetic Pyrethroid Insecticides:

acrinathrin, allethrin, benfluthrin, beta-cyfluthrin, bifenthrin, cycloprothrin, cyfluthrin, cyhalothrin, cypermethrin, deltamethrin, esfenvalerate, etofenprox, fenpropathrin, fenvalerate, flucythrinate, flufenprox, flumethrin, fluvalinate, halfenprox, imiprothrin, permethrin, prallethrin, pyrethrins, resmethrin, sigma-cypermethrin, silafluofen, tefluthrin, tralomethrin, transfluthrin, 2,3,5,6-tetrafluoro-4-(methoxymethyl)benzyl (EZ)-(1RS, 3RS; 1RS, 3SR)-2,2-dimethyl-3-prop-1-enylcyclopropanecarboxylate, 2,3,5,6-tetrafluoro-4-methylbenzyl (EZ)-(1RS, 3RS; 1RS, 3SR)-2,2-dimethyl-3-prop-1-enylcyclopropanecarboxylate, 2,3,5,6-tetrafluoro-4-(methoxymethyl)benzyl (1RS, 3RS; 1RS, 3SR)-2,2-dimethyl-3-(2-methyl-1-propenyl)cyclopropanecarboxylate, and the like;

(4) Nereistoxin Compounds:

cartap, bensultap, thiocyclam, monosultap, bisultap, and the like;

(5) Neonicotinoid Compounds:

imidacloprid, nitenpyram, acetamiprid, thiamethoxam, thiacloprid, and the like;

(6) Benzoylurea Compounds:

chlorfluazuron, bistrifluoron, diafenthiuron, diflubenzuron, fluazuron, flucycloxuron, flufenoxuron, hexaflumuron, lufenuron, novaluron, noviflumuron, teflubenzuron, triazuron, triflumuron, and the like;

(7) Phenylpyrazole Compounds:

acetoprole, fipronil, vaniliprole, pyriprole, pyrafluprole, and the like;

(8) Bt Toxin Insecticides:

live spores-derived from and crystal toxins produced from Bacillus thuringiesis and a mixture thereof;

(9) Hydrazine Compounds:

chromafenozide, halofenozide, methoxyfenozide, tebufenozide, and the like;

(10) Organic Chlorine Compounds:

aldrin, dieldrin, dienochlor, endosulfan, methoxychlor, and the like;

(11) Natural Insecticides:

machine oil, nicotine sulfate, and the like;

(12) Other Insecticides:

ivermectin-B, cyenopyrafen, bromopropylate, buprofezin, chlorphenapyr, cyenopyrafen, cyromazine, D-D (1,3-dichloropropane), emamectin-benzoate, fenazaquin, flupyrazofos, hydroprene, indoxacarb, lepimectin, metoxadiazone, milbemycin-A, pymetrozine, pyridalyl, pyriproxyfen, spinosad, sulfluramid, tolfenpyrad, triazamate, flubendiamide, cyflumetofen, arsenic acid, benclothiaz, calcium cyanamide, calcium polysulfide, chlordane, DDT, DSP, flufenerim, flonicamid, flurimfen, formetanate, metam-ammonium, metam-sodium, methyl bromide, nidinotefuran, potassium oleate, protrifenbute, spiromesifen, sulfur, metaflumizone, spirotetramat, pyrifluquinazon, tralopyril, a compound represented by the following formula (A):

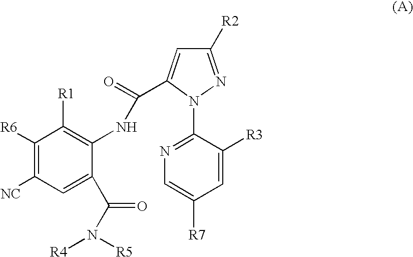

wherein R¹ represents Me, Cl, Br or F, R² represents F, Cl, Br, C1-C4 haloalkyl or C1-C4 haloalkoxy, R³ represents F, Cl or Br, R⁴ represents H, or C1-C4 alkyl, C3-C4 alkenyl, C3-C4 alkynyl, C3-C5 cycloalkyl or C4-C6 cycloalkylalkyl which may be substituted with one or more substituents selected from the group consisting of a halogen atom, CN, SMe, S(O)Me, S(O)₂Me and OMe, R⁵ represents H or Me, R⁶ represents H, F or Cl, and R⁷ represents H, F or Cl; and the like.

Examples of the acaricide include acequinocyl, amitraz, benzoximate, bifenazate, bromopropylate, chinomethionat, chlorobezilate, CPCBS (chlorfenson), clofentezine, cyenopyrafen, cyflumetofen, kelthane (dicofol), etoxazole, fenbutatin oxide, fenothiocarb, fenpyroximate, fluacrypyrim, fluproxyfen, hexythiazox, propargite (BPPS), polynactins, pyridaben, pyrimidifen, tebufenpyrad, tetradifon, spirodiclofen, spiromesifen, spirotetramat, amidoflumet, and the like.

Examples of the nematicide include DCIP, fosthiazate, levamisol hydrochloride, methylisothiocyanate, morantel tartarate, imicyafos, and the like.

Examples of the fungicide include acibenzolar-S-methyl, amobam, amisulbrom, ampropylfos, anilazine, azoxystrobin, benalaxyl, benodanil, benomyl, benthiavalicarb, benthiazole, benthoxazin, bitertanol, blasticidin-S, Bordeaux mixture, boscalid, bromuconazole, buthiobate, calcium hypochlorite, calcium polysulfide, captan, carbendazol, carboxin, carpropamid, chlobenthiazone, chloroneb, chloropicrin, chlorothalonil (TPN), chlorthiophos, cinnamaldehyde, clozylacon, CNA (2,6-dichloro-4-nitroaniline), copper hydroxide, copper sulfate, cyazofamid, cyfluphenamid, cymoxanil, cyproconazole, cyprodinil, cyprofuram, dazomet, debacarb, dichlofluanid, D-D (1,3-dichloropropane), diclocymet, diclomezine, diethofencarb, difenoconazole, diflumetorim, dimefluazole, dimethirimol, dimethomorph, diniconazole-M, dinocap, edifenphos, enestroburin, epoxiconazole, nickel dimethyldithiocarbamate, etaconazole, ethaboxam, ethirimol, etridiazole, famoxadone, fenamidone, fenarimol, fenbuconazole, fendazosulam, fenhexamid, fenoxanil, fenpiclonil, fenpropidin, fenpropimorph, fentiazon, fentin hydroxide, ferimzone, fluazinam, fludioxonil, flumetover, flumorph, fluoroimide, fluotrimazole, fluoxastrobin, fluquinconazole, flusilazole, flusulfamide, flutolanil, flutriafol, fosetyl-Al, phthalate, fuberidazole, furalaxyl, furametpyr, furcarbanil, furconazole-cis, hexaconazole, hymexazol, IBP, imazalil, imibenconazole, iminoctadine-albesilate, iminoctadine-triacetate, iodocarb, ipconazole, iprodione, iprovalicarb, isoprothiolane, kasugamycin, kresoxim-methyl, mancozeb, mandipropamid, maneb, mepanipyrim, mepronil, meptyldinocap, metalaxyl, metalaxyl-M, metam-sodium, methasulfocarb, methyl bromide, metconazole, methfuroxam, metominostrobin, metrafenone, metsulfovax, mildiomycin, milneb, myclobutanil, myclozolin, nabam, orysastrobin, ofurace, oxadixyl, oxolinic acid, oxpoconazole, oxycarboxin, oxytetracycline, pefurazoate, penconazole, pencycuron, picoxystrobin, polycarbamate, polyoxin, potassium hydrogen carbonate, probenazole, prochloraz, procymidone, propamocarb-hydrochloride, propiconazole, propineb, proquinazid, prothiocarb, prothioconazole, pyracarbolid, pyraclostrobin, pyrazophos, pyributicarb, pyrifenox, pyrimethanil, pyroquilon, quinoxyfen, quintozene (PCNB), silthiopham, simeconazole, sipconazole, sodium bicarbonate, sodium hypochlorite, spiroxamine, SSF-129 ((E)-2-[2-(2,5-dimethylphenoxymethyl)phenyl]-2-methoxyimino-N-methylacetamide), streptomycin, sulfur, tebuconazole, tecloftalam, tetraconazole, thiabendazole, thiadinil, thiram (TMTD), thifluzamide, thiophanate-methyl, tolclofos-methyl, TPN, triadimefon, triadimenol, triazoxide, triclamide, tricyclazole, tridemorph, triflumizole, trifloxystrobin, triforine, triticonazole, validamycin, vinclozolin, viniconazole, zineb, ziram, zoxamide, and the like.

Examples of the herbicide and/or the plant growth regulating substance include abscisic acid, acetochlor, acifluorfen-sodium, alachlor, alloxydim, ametryn, amicarbazone, amidosulfuron, aminoethoxyvinylglycine, aminopyralid, AC94, 377, amiprofos-methyl, ancymidol, asulam, atrazine, aviglycine, azimsulfuron, beflubutamid, benfluralin, benfuresate, bensulfuron-methyl, bensulide (SAP), bentazone, benthiocarb, benzamizole, benzfendizone, benzobicyclon, benzofenap, benzyl adenine, benzylaminopurine, bialaphos, bifenox, brassinolide, bromacil, bromobutide, butachlor, butafenacil, butamifos, butylate, canfestrole, calcium carbonate, calcium peroxide, carbaryl, chlomethoxynil, chloridazon, chlorimuron-ethyl, chlorphthalim, chlorpropham, chlorsulfuron, chlorthal-dimethyl, chlorthiamid (DCBN), choline chloride, cinidon-ethyl, cinmethylin, cinosulfuron, clethodim, clomeprop, cloxyfonac-sodium, chlormequat chloride, 4-CPA (4-chlorophenoxyacetic acid), cliprop, clofencet, cumyluron, cyanazine, cyclanilide, cyclosulfamuron, cyhalofop-butyl, 2,4-D salt (2,4-dichlorophenoxyacetic acid salts), dichlorprop (2,4-DP), daimuron, dalapon (DPA), dimethenamid-P, daminozide, dazomet, n-decyl alcohol, dicamba-sodium (MDBA), dichlobenil (DBN), diflufenican, dikegulac, dimepiperate, dimethametryn, dimethenamid, diquat, dithiopyr, diuron, endothal, epocholeone, esprocarb, ethephon, ethidimuron, ethoxysulfuron, ethychlozate, etobenzanid, fenarimol, fenoxaprop-ethyl, fentrazamide, flazasulfuron, florasulam, fluazifop-butyl, fluazolate, flucarbazone, flucetosulfuron, flufenacet, flufenpyr, flumetralin, flumioxazine, flupropanate-sodium, flupyrsulfuron-methyl-sodium, flurprimidol, fluthiacet-methyl, foramsulfuron, forchlorfenuron, fomesafen, gibberelin, glufosinate, glyphosate, halosulfuron-methyl, hexazinone, imazamox, imazapic, imazapyr, imazaquin, imazosulfuron, inabenfide, indole acetic acid (IAA), indole butyric acid, iodosulfuron, ioxynil-octanoate, isouron, isoxachlortole, isoxadifen, karbutilate, lactofen, lenacil, linuron, LGC-42153, maleic hydrazide, mecoprop (MCPP), MCP salts (2-methyl-4-chlorophenoxyacetic acid salts), MCPA-thioethyl, MCPB (2-methyl-4-chlorophenoxybutanoic acid ethyl ester), mefenacet, mefluidide, mepiquat, mesosulfuron, mesotrione, methyl daimuron, metolachlor, metribuzin, metsulfuron-methyl, molinate, naphthylacetic acid, NAD (1-naphthaleneacetamide), naproanilide, napropamide, n-decyl alcohol, nicosulfuron, n-phenylphthalamic acid, orbencarb, orthosulfanuron, oxadiazon, oxaziclomefone, oxine-sulfate, paclobutrazol, paraquat, pelargonic acid, pendimethalin, pentoxazone, pethoxamide, phenmedipham, picloram, picolinafen, pinoxaden, piperonyl butoxide, piperophos, pretilachlor, primisulfuron-methyl, procarbazine, prodiamine, profluazol, profoxydim, prohexadione-calcium, prohydrojasmon, prometryn, propanil, propoxycarbazone, propyrzamide, pyraflufen-ethyl, pyrasulfotole, pyrazolate, pyrazosulfuron-ethyl, pyrazoxyfen, pyribenzoxim, pyributicarb, pyridafol, pyridate, pyriftalid, pyriminobac-methyl, pyrithiobac, quiclorac, quinoclamine, quizalofop-ethyl, rimsulfuron, sethoxydim, siduron, simonize, simetryn, sodium chlorate, sulfosufuron, swep (MCC), tebuthiuron, tefurytrione, tembotrione, tepraloxydim, terbacil, terbucarb (MBPMC), thenylchlor, thiazafluoron, thidiazuron, thiencarbazone methyl, thifensulfuron-methyl, triaziflam, tribufos, triclopyr, tridiphane, trifloxysulfuron, trifluralin, trinexapac-ethyl, tritosulfuron, uniconazole-P, vemolate (PPTC), flucetosulfuron, orthosulfanuron, pinoxaden, pyrasulfotole, tefuryltrione, tembotrione, thiencarbazone methyl, and the like.

Examples of the synergist include piperonyl butoxide, sesamex, sulfoxide, N-(2-ethylhexyl)-8,9,10-trinorborn-5-ene-2,3-dicarboximide (MGK 264), WARF-antiresistant, diethyl maleate, DMC, FDMC, ETP, and ETN.

Examples of the agent for alleviating chemical injuries include allidochlor, benoxacor, cloquintocet-mexyl, cyometrinil, cyprosulfamide, daimuron, dichlormid, fenchlorazole-ethyl, fenclorim, flurazole, fluxofenim, furilazole, isoxadifen-ethyl, mefenpyr-diethyl, MG191, naphthalic anhydride, oxabetrinil, and 1,8-naphthalic anhydride.

In the case of plants grown via a seedling raising stage, such as fruit vegetables including eggplant and tomato and leaf vegetables including cabbage and lettuce, there are a seedling raising stage, a seedling stage, a transplanting stage in an agricultural field, and a growing stage in an agricultural field. In the case of plants of which seeds are usually directly sowed in an agricultural field, such as cotton, corn and soybean, there are a seed stage, a sowing stage in an agricultural field, and a growth stage in an agricultural field. According to the method of the present invention, the composition for soil treatment of the present invention can be applied to soil where a plant is grown at any stage of plant cultivation.

Examples of a plant suitable for the method of the present invention include the following crop plants: creeping bent grass (Agrostis stronifera), onion (Allium cepa), pineapple (Ananas comosus), peanut (Arachis hypogaca), asparagus (Asparagus officinalis), Beta vulgaris var. altissima, Beta vulgaris var. rapa, Chinese cabbage (Brassica campestris), cabbage (Brassica oleracea), Brassica napus var. napus, Brassica napus var. napobrassica, Brassica rapa var. silvestris, rapeseed (Brassica rapa), tea (Camelliasinensis), capsicum (Capsicum annuum), safflower (Carthamus tinctorious), hickory (Carya illinoinesis), lemon (Citrus limon), orange (Citrus sinensis), Arabian coffee (Coffea arabica), robusta coffee (Coffea canephora), Liberian coffee (Coffea liberica), cucumber (Cucumis sativus), Bermuda grass (Cynodon dactylon), carrot seed (Daucus carota), oil palm (Elaeis guineensis), fescue (Festuca arundinacea), wild strawberry (Fragaria vesca), soybean (Glycine max), upland cotton (Gossypium hirsutum), Indian cotton (Gossypium arboreum), Asian cotton (Gossypium herbaceum), Sea Island cotton (Gossypium vitifolium), sunflower (Helianthus annuus), Para rubber tree (Hevea brasiliensis), barley (Hordeum vulgare), hop (Humulus lupulus), sweet potato (Ipomoea batatas), walnut (Juglans regia), lettuce (Lactuca sativa), lentil (Lens culinaris), flax (Linum usitatissimum), ryegrass (Lolium perenne), tomato (Lycopersicon lycopersicum), apple (Malus spec.), cassava (Manihot esculenta), alfalfa (Medicago sativa), Musa spec., tobacco (Nocotiana tabacum) (N. rustica), olive (Olea europaea), rice plant (Oryza sativa), lima bean (Phaseolus lunatus), common bean (Phaseolus vulgaris), Norway spruce (Picea abies), pine (Pinus spec.), pea (Pisum sativum), bluegrass (Poa pratensis), cherry (Prunus avium), peach (Prunus persica), pear (La France) (Pyrus communis), Chinese radish (Raphanus sativus), currant (Ribes sylvestre), castor-oil plant (Ricinus communis), sugarcane (Saccharum officinarum), rye (Secale cereale), eggplant (Solanum melongena), potato (Solanum tuberosum), common sorghum (Sorghum bicolor) [kaoliang (S. vulgare)], cacao (Theobroma cacao), red clover (Trifolium pratense), wheat (Triticum aestivum), durum wheat (Triticum durum), broad bean (Vicia faba), grape (Vitis vinifera), corn (Zea mays), and Japanese lawngrass (Zoysia japonica).

The aforementioned crop plants include those to which resistance to herbicides has been imparted by a classical breeding method, a genetic engineering technique or the like. When an HPPD inhibitor such as isoxaflutole; an ALS inhibitor such as imazethapyr or thifensulfuron-methyl; an EPSP synthesizing enzyme inhibitor; a glutamine synthesizing enzyme inhibitor; an acetyl CoA carboxylase inhibitor; or a herbicide such as bromoxynil is applied to the crop plant to which resistance to a herbicide has been imparted, the chemical has no harmful effect on the crop plant.

Examples of the crop plant to which resistance to a herbicide has been imparted by a classical breeding method include Clearfield (registered trademark) canola to which resistance to a imidazolinone herbicide has been imparted; STS soybean to which resistance to a sulfonylurea herbicide has been imparted; and SR corn to which resistance to an acetyl CoA carboxylase inhibitor has been imparted. For example, crop plants to which resistance to acetyl CoA carboxylase inhibitors has been imparted are found in Proc. Natl. Acad. Sci. USA 1990, 87, 7175.

In addition, a mutant acetyl CoA carboxylase which provides resistance to an acetyl CoA carboxylase inhibitor is known, for example, in Weed Science 53:728-746, 2005. When a gene encoding the mutant acetyl CoA carboxylase is introduced into a crop plant by a genetic engineering technique or when a mutation related to impartation of acetyl CoA carboxylase resistance is introduced into a gene encoding acetyl CoA carboxylase of a crop plant, resistance to an acetyl CoA carboxylase inhibitor herbicide may be imparted to the crop plant.

Examples of the crop plant to which resistance to a herbicide has been imparted by a genetic engineering technique include corn cultivars to which resistance to glyphosate and glufosinate has been imparted. Some of such corn cultivars are sold under the trade name of RoundupReady (registered trademark) and LibertyLink (registered trademark).

The aforementioned crop plants include those to which ability to produce an insecticidal toxin has been imparted by a genetic engineering technique.

Examples of the insecticidal toxin include insecticidal proteins derived from Bacillus cereus and Bacillus popilliae; d-endotoxins derived from Bacillus thuringiensis, such as Cry1Ab, Cry1Ac, Cry1F, Cry1Fa2, Cry2Ab, Cry3A, Cry3Bb1 and Cry9C; insecticidal proteins derived from Bacillus thuringiensis, such as VIP 1, VIP 2, VIP 3 and VIP 3A; insecticidal proteins derived from nematodes; toxins produced by animals such as scorpion toxins, spider toxins, bee toxins and insect-specific nerve toxins; fungal toxins; plant lectin; agglutinin; protease inhibitors such as trypsin inhibitors, serine protease inhibitors, patatin, cystatin, and papain inhibitors; ribosome-inactivating proteins (RIP) such as ricin, corn-RIP, abrin, saporin, and briodin; steroid metabolizing enzymes such as 3-hydroxysteroid oxidase, ecdysteroid-UDP-glucosyltransferase, and cholesterol oxidase; ecdysone inhibitors; HMG-COA reductase; ion channel inhibitors such as sodium channel inhibitors and calcium channel inhibitors; juvenile hormone esterase; diuretic hormone receptors; stilbene synthase; dibenzyl synthase; chitinase; and glucanase.

The insecticidal toxin includes hybrid proteins of the aforementioned insecticidal proteins, and proteins in which a part of amino acids constituting the aforementioned insecticidal proteins is deleted or substituted. The hybrid protein is made by combining different domains of the aforementioned insecticidal proteins by a genetic engineering technique. An example of the toxin in which a part of amino acids constituting the aforementioned insecticidal protein is deleted includes Cry1Ab in which a part of amino acids is deleted.

The insecticidal toxin, and the crop plant to which ability to produce the insecticidal toxin has been imparted by a genetic engineering technique are described, for example, in EP-A-0 374 753, WO 93/07278, WO 95/34656, EP-A-0 427 529, EP-A-451878, and WO 03/052073.

The crop plant to which ability to produce an insecticidal toxin has been imparted by a genetic engineering technique has resistance to attack by a pest such as a coleopteran pest, dipteran pest and/or a lepidopteran pest.

Among the crop plants to which ability to produce an insecticidal toxin has been imparted by a genetic engineering technique, examples of commercially available crop plant include YieldGard (registered trademark) (a corn cultivar expressing Cry1Ab toxin), YieldGard Rootworm (registered trademark) (a corn cultivar expressing Cry3Bb1 toxin), YieldGard Plus (registered trademark) (a corn cultivar expressing Cry1Ab and Cry3Bb1 toxins), Herculex I (registered trademark) (a corn cultivar expressing Cry1Fa2 toxin and phosphinotricin N-acetyltransferase (PAT) for imparting resistance to gluphosinate), NuCOTN33B (registered trademark) (a cotton cultivar expressing Cry1Ac toxin), Bollgard I (registered trademark) (a cotton cultivar expressing Cry1Ac toxin), Bollgard II (registered trademark) (a cotton cultivar expressing Cry1Ac and Cry2Ab toxins), VIPCOT (registered trademark) (a cotton cultivar expressing VIP toxin), NewLeaf (registered trademark) (a potato cultivar expressing Cry3A toxin), NatureGard (registered trademark), Agrisure (registered trademark), GT Advantage (GA21 glyphosate-resistance character), Agrisure (registered trademark), CB Advantage (Btl1 corn borer (CB) character), and Protecta (registered trademark).

The aforementioned crop plants include those to which ability to produce an anti-pathogen substance has been imparted by a genetic engineering technique.

The anti-pathogen substance includes anti-pathogen substances which are usually produced by microorganisms, for example, PR proteins (PRPs described in EP-A-0 392 225); ion channel inhibitors such as sodium channel inhibitors, and calcium channel inhibitors (e.g. KP1, KP4, KP6 toxins etc. produced by viruses); stilbene synthase; dibenzyl synthase; chitinase; glucanase; peptide antibiotics, and heterocycle-containing antibiotics; proteins involved in plant disease-resistance (described in WO 03/000906); and the like.

Examples of the crop plant to which ability to produce an anti-pathogen substance has been imparted by a genetic engineering technique include plants described in EP-A-0 392 225, WO 05/33818, and EP-A-0 353 191.

Examples of a pest which damages the aerial part of a plant and can be controlled by the method of the present invention include pests (e.g. harmful insects and harmful mites) which damage the aerial part of a plant by eating, sucking or the like, and specific examples thereof include pests as mentioned below.

Hemiptera: Planthoppers (Delphacidae) such as small brown planthopper (Laodelphax striatellus), brown rice planthopper (Nilaparvata lugens), and white-backed rice planthopper (Sogatella furcifera); leafhoppers (Deltocephalidae) such as green rice leafhopper (Nephotettix cincticeps), and green rice leafhopper (Nephotettix virescens); aphids (Aphididae) such as cotton aphid (Aphis gossypii), green peach aphid (Myzus persicae), cabbage aphid (Brevicoryne brassicae), potato aphid (Macrosiphum euphorbiae), foxglove aphid (Aulacorthum solani), oat bird-cherry aphid (Rhopalosiphum padi), and tropical citrus aphid (Toxoptera citricidus); stink bugs (Pentatomidae) such as green stink bug (Nezara antennata), bean bug (Riptortus clavetus), rice bug (Leptocorisa chinensis), white spotted spined bug (Eysarcoris parvus), stink bug (Halyomorpha mista), and tarnished plant bug (Lygus lineolaris); whiteflies (Aleyrodidae) such as greenhouse whitefly (Trialeurodes vaporariorum), and silver leaf whitefly (Bemisia argentifolii); scales (Coccidae) such as California red scale (Aonidiella aurantii), San Jose scale (Comstockaspis perniciosa), citrus north scale (Unaspis citri), red wax scale (Ceroplastes rubens), and cottonycushion scale (Icerya purchasi); lace bugs (Tingidae); psyllids (Psyllidae); etc.

Lepidoptera: Pyralid moths (Pyralidae) such as rice stem borer (Chilo suppressalis), yellow rice borer (Tryporyza incertulas), rice leafroller (Cnaphalocrocis medinalis), cotton leafroller (Notarcha derogata), Indian meal moth (Plodia interpunctella), oriental corn borer (Ostrinia furnacalis), cabbage webworm (Hellula undalis), and bluegrass webworm (Pediasia teterrellus); owlet moths (Noctuidae) such as common cutworm (Spodoptera litura), beet armyworm (Spodoptera exigua), armyworm (Pseudaletia separata), cabbage armyworm (Mamestra brassicae), black cutworm (Agrotis ipsilon), beet semi-looper (Plusia nigrisigna), Thoricoplusia spp., Heliothis spp., and Helicoverpa spp.; white butterflies (Pieridae) such as common white (Pieris rapae); tortricid moths (Tortricidae) such as Adoxophyes spp., oriental fruit moth (Grapholita molesta), soybean pod borer (Leguminivora glycinivorella), azuki bean podworm (Matsumuraeses azukivora), summer fruit tortrix (Adoxophyes orana fasciata), smaller tea tortrix (Adoxophyes sp.), oriental tea tortrix (Homona magnanima), apple tortrix (Archips fuscocupreanus), and codling moth (Cydia pomonella); leafblotch miners (Gracillariidae) such as tea leafroller (Caloptilia theivora), and apple leafminer (Phylionorycter ringoneella); Carposimidae such as peach fruit moth (Carposina niponensis); lyonetiid moths (Lyonetiidae) such as Lyonetia spp.; tussock moths (Lymantriidae) such as Lymantria spp., and Euproctis spp.; yponomeutid moths (Yponomeutidae) such as diamondback moths (Plutella xylostella); gelechiid moths (Gelechiidae) such as pink bollworm (Pectinophora gossypiella), and potato tubeworm (Phthorimaea operculella); tiger moths and allies (Arctiidae) such as fall webworm (Hyphantria cunea); tineid moths (Tineidae) such as casemaking clothes moth (Tinea translucens), and webbing clothes moth (Tineola bisselliella); etc.

Thysanoptera: Thrips (Thripidae) such as yellow citrus thrip (Frankliniella occidentalis), melon thrip (Thrips palmi), yellow tea thrip (Scirtothrips dorsalis), onion thrip (Thrips tabaci), flower thrip (Frankliniella intonsa), tobacco thrip (Frankliniella fusca), etc.

Diptera: Onion maggot (Hylemya antiqua), seedcorn maggot (Hylemya platura), Anopheles sinensis, rice leafminer (Agromyza oryzae), rice leafminer (Hydrellia griseola), rice stem maggot (Chlorops oryzae), melon fly (Dacus cucurbitae), Mediterranean fruit fly (Ceratitis capitata), legume leafminer (Liriomyza trifolii), etc.

Coleoptera: Twenty-eight-spotted ladybird (Epilachna vigintioctopunctata), cucurbit leaf beetle (Aulacophora femoralis), striped flea beetle (Phyllotreta striolata), rice leaf beetle (Oulema oryzae), rice curculio (Echinocnemus squameus), rice water weevil (Lissorhoptrus oryzophilus), boll weevil (Anthonomus grandis), azuki bean weevil (Callosobruchus chinensis), hunting billbug (Sphenophorus venatus), Japanese beetle (Popillia japonica), cupreous chafer (Anomala cuprea), corn root worms (Diabrotica spp.), Colorado beetle (Leptinotarsa decemlineata), click beetles (Agriotes spp.), cigarette beetle (Lasioderma serricorne), varied carper beetle (Anthrenus verbasci), red flour beetle (Tribolium castaneum), powder post beetle (Lyctus brunneus), white-spotted longicorn beetle (Anoplophora malasiaca), pine shoot beetle (Tomicus piniperda), etc.

Orthoptera: Asiatic locust (Locusta migratoria), African mole cricket (Gryllotalpa africana), rice grasshopper (Oxya yezoensis), rice grasshopper (Oxya japonica), etc.

Hymenoptera: Cabbage sawfly (Athalia rosae), leaf-cutting ant (Acromyrmex spp.), fire ant (Solenopsis spp.), etc.

Acarina: Spider mites (Tetranychidae) such as two-spotted spider mite (Tetranychus urticae), citrus red mite (Panonychus citri), and Oligonychus spp.; eriophyid mites (Eriophyidae) such as pink citrus rust mite (Aculops pelekassi); tarosonemid mites (Tarsonemidae) such as broad mite (Polyphagotarsonemus latus); false spider mites (Tenuipalpidae); etc.

The method of the present invention is preferably used for protection from lepidopterans and thrips.

### EXAMPLES

Then, the present invention is explained in more detail by reference to Examples to which the present invention is not limited.

First, synthesis examples of the present compound are shown below.

### Synthesis Example 1

### 3-(2,6-Difluorobenzoyl)-1-[2-fluoro-4-(1,1,2,2-tetrafluoroethoxy)phenyl]-1-methylurea (hereinafter, referred to as the present compound (1))

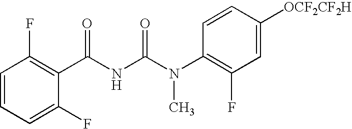

To a solution of 2-fluoro-N-methyl-4-(1,1,2,2-tetrafluoroethoxy)aniline (1.00 g) in diethyl ether (4.0 ml) was added a solution of 2,6-difluorobenzoyl isocyanate (0.75 g) in diethyl ether (1.0 ml) under ice-cooling. The mixture was stirred at room temperature for 2 hours, and then hexane (10 ml) was added thereto. The mixture was filtered, and a solid on the filter was dried to obtain 1.58 g of the present compound (1).

### Present Compound (1)

¹H-NMR (DMSO-d₆) δ (ppm): 3.20 (3H, s), 6.67-6.93 (1H, m), 7.09-7.20 (3H, m), 7.33-7.36 (1H, m), 7.45-7.53 (2H, m), 10.77 (1H, brs)

### Synthesis Example 2

### 3-(2,6-Difluorobenzoyl)-1-(4-trifluoromethoxyphenyl)-1-methylurea (hereinafter, referred to as the present compound (2))

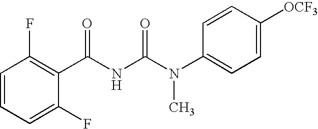

To a solution of N-methyl-4-trifluoromethoxyaniline (0.60 g) in diethyl ether (2.4 ml) was added a solution of 2,6-difluorobenzoyl isocyanate (0.57 g) in diethyl ether (0.5 ml) under ice-cooling. The mixture was stirred at room temperature for 2 hours, and then hexane (0.6 ml) was added thereto. The mixture was filtered, and a solid on the filter was dried to obtain 1.13 g of the present compound (2).

### Present Compound (2)

¹H-NMR (DMSO-d₆) δ (ppm): 3.25 (3H, s), 7.12-7.16 (2H, m), 7.41-7.55 (5H, m), 10.68 (1H, brs).

### Synthesis Example 3

### 3-(2,6-Difluorobenzoyl)-1-(4-chlorophenyl)-1-methylurea (hereinafter, referred to as the present compound (3))

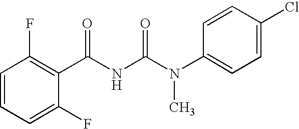

The present compound (3) is a compound described in DE 2123236 and can be prepared by the same method as described therein.

### Synthesis Example 4

### 3-(2,6-Difluorobenzoyl)-1-(4-chlorophenyl)-1,3-dimethylurea (hereinafter, referred to as the present compound (4))

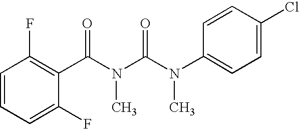

The present compound (4) is a compound described in JP-A 2-3659 and can be prepared by the same method as described therein.

### Synthesis Example 5

### 3-(2,6-Difluorobenzoyl)-1-(3,5-dichloro-2,4-difluorophenyl)-1,3-dimethylurea (hereinafter, referred to as the present compound (5))

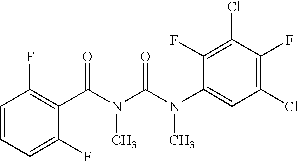

The present compound (5) is a compound described in JP-A 2-3659 and can be prepared by the same method as described therein.

### Synthesis Example 6

### 3-(2,6-Difluorobenzoyl)-1-[2-fluoro-4-(1,1,2,2-tetrafluoroethoxy)phenyl]-1,3-dimethylurea (hereinafter, referred to as the present compound (6))

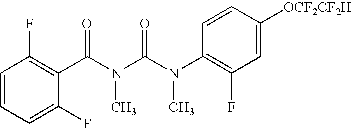

The present compound (6) is a compound described in JP-A 4-26667 and can be prepared by the same method as described therein.

### Synthesis Example 7

### 3-(2,6-Difluorobenzoyl)-1-[2-fluoro-4-(trifluoromethyl)phenyl]-1,3-dimethylurea (hereinafter, referred to as the present compound (7))

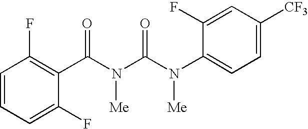

To a solution of 3-(2,6-difluorobenzoyl)-1-[2-fluoro-4-(trifluoromethyl)phenyl]-1-methylurea (1.01 g) in 1-methyl-2-pyrrolidone (10.0 ml) was added sodium hydride (128 mg) at about 2° C. The mixture was stirred for 30 minutes, and then methyl iodide (0.40 ml) was added thereto at 2° C. The mixture was stirred at 2 to 3° C. for 3 hours. Then, to the reaction mixture was added a mixture of an aqueous saturated ammonium chloride solution (10 ml) and water (10 ml) under ice-cooling, followed by extraction with ethyl acetate (20 ml) three times. Organic layers were combined, washed with an aqueous saturated sodium chloride solution three times, dried over anhydrous magnesium sulfate, and then concentrated under reduced pressure. The resulting residue was subjected to silica gel column (eluting with ethyl acetate:chloroform:hexane=1:1:4) to obtain 0.79 g of the present compound (7).

### Present Compound (7)

¹H-NMR (DMSO, 80° C.) δ (ppm): 3.07 (3H, s), 3.28 (3H, s), 7.09-7.13 (2H, m), 7.50-7.60 (3H, m), 7.69-7.71 (1H, m).

### Synthesis Example 8

### 3-(2,6-Difluorobenzoyl)-1-[2-fluoro-4-(trifluoromethyl)phenyl]-1-methylurea (hereinafter, referred to as the present compound (8))

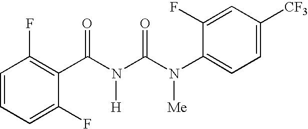

According to the same manner as that of Synthesis Example 1, the present compound (8) was prepared.

### Present Compound (8)

¹H-NMR (DMSO) δ (ppm): 3.24 (3H, s), 7.12-7.16 (2H, m), 7.47-7.49 (1H, m), 7.51-7.67 (2H, m), 7.81-7.84 (1H, m), 10.89 (1H, brs).

### Synthesis Example 9

### 3-(2,6-Difluorobenzoyl)-1-[4-(1,1,2,2-tetrafluoroethoxy)phenyl]-1-methylurea (hereinafter, referred to as the present compound (9))

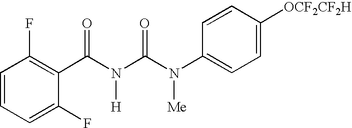

According to the same manner as that of Synthesis Example 1, the present compound (9) was prepared.

### Present Compound (9)

¹H-NMR (CDCl₃) δ (ppm): 3.22 (3H, s), 5.81-6.09 (1H, m) 6.92-7.05 (2H, m), 7.32-7.55 (6H, m).

Then, examples of the composition for soil treatment of the present invention are shown. The term “part(s)” means “part(s) by weight”.

### Preparation Example 1

Any one (10 parts) of the present compounds (1) to (9) is dissolved in a mixture of xylene (35 parts) and N,N-dimethylformamide

(35 parts). To the solution are added polyoxyethylene styryl phenyl ether (14 parts) and calcium dodecylbenzenesulfonate (6 parts). The mixture is stirred well to obtain a 10% emulsion concentrate.

### Preparation Example 2

Any one (20 parts) of the present compounds (1) to (9) is added to a mixture of sodium lauryl sulfate (4 parts), calcium ligninsulfonate (2 parts), synthetic hydrous silicon oxide fine powder (20 parts) and diatomaceous earth (54 parts). The mixture is stirred well to obtain a 20% wettable powder.

### Preparation Example 3

To any one (2 parts) of the present compounds (1) to (9) are added synthetic hydrous silicon oxide fine powder (1 part), calcium ligninsulfonate (2 parts), bentonite (30 parts) and kaolin clay (65 parts). The mixture is stirred well, and then an appropriate amount of water is added thereto. The mixture is further stirred, granulated with a granulator, and then dried with ventilation to obtain a 2% granule.

### Preparation Example 4

Any one (10 parts) of the present compounds (1) to (9), white carbon (35 parts) containing a polyoxyethylene alkyl ether sulfate ammonium salt (50 parts), and water (55 parts) are mixed and then finely ground by a wet grinding method to obtain a 10% suspension concentrate.

Then, examples of the method of the present invention are shown.

In the following Test Examples, an inhibiting effect on damage of the aerial part of a plant by a pest (damage-inhibiting effect) was calculated by the following equation. Damage-Inhibiting Effect (%)=[1-{(Extent of feeding damage in treated section)/(Extent of feeding damage in untreated section)}]×100

### Test Example 1

The present compounds (1) and (6) (each 5 mg) were dissolved in a mixture (0.1 ml) of Solgen TW-20 (manufactured by Dai-Ichi Kogyo Seiyaku Co., Ltd.) and acetone (mixing volume ratio; Solgen TW-20:acetone=1:19), and then diluted with ion-exchanged water (5 ml) to prepare a test water dilution of each test compound.

Seeds of cabbage (Brassicae oleracea) were sowed in a plug cell tray (each cell volume: 27 ml, depth: 5.0 cm) filled with nursery soil, and the test water dilution (5 ml) was drenched on the soil surface of the seeded cell. The plant was raised until a 2.5-leaf stage. The root part of the raised cabbage seedling was removed and the stem and leaf part was placed in a polyethylene cup (volume: 180 ml). Into the cup, 10 second-instar larvae of a diamondback moth (Plutella xylostella) were released. After the cup was stored in the chamber at 25° C. for 7 days, the extent of feeding damage of the cabbage seedling by diamondback moth larvae was examined. The damage extent of the cabbage seedling grown in soil which was treated with a test compound (treated section) was compared with the damage extent of the cabbage seedling grown in soil which was not treated with a test compound (non-treated section), and a damage-inhibiting effect was calculated by the aforementioned equation.

As a result, in a section treated with the present compound (1) and a section treated with the present compound (6), the damage-inhibiting effect was 90% or more, and thus, the aerial part of a cabbage seedling could be protected from damage by a diamondback moth.

### Test Examples 2

The present compounds (3), (8) and (9) (each 5 mg) were dissolved in a mixture (0.1 ml) of Solgen TW-20 (manufactured by Dai-Ichi Kogyo Seiyaku Co., Ltd.) and acetone (mixing volume ratio; Solgen TW-20:acetone=1:19), and then diluted with ion-exchanged water (5 ml) to prepare a test water dilution of each test compound.

Seeds of cabbage (Brassicae oleracea) were sowed in a plug cell tray (each cell volume: 27 ml, depth: 5.0 cm) filled with nursery soil, and the test water dilution (5 ml) was drenched on the soil surface of the seeded cell. The plant was raised until a 2.5-leaf stage. The root part of the raised cabbage seedling was removed and the stem and leaf part was placed in a polyethylene cup (volume: 180 ml). Into the cup, 10 first-instar larvae of a common cutworm (Spodoptera litura) were released. After the cup was stored in the chamber at 25° C. for 7 days, the extent of feeding damage of the cabbage seedling by common cutworm larvae was examined. The damage extent of the cabbage seedling grown in soil which was treated with a test compound (treated section) was compared with the damage extent of the cabbage seedling grown in soil which was not treated with a test compound (non-treated section), and a damage-inhibiting effect was calculated by the aforementioned equation.

As a result, in a section treated with the present compound (3), a section treated with the present compound (8) and a section treated with the present compound (9), the damage-inhibiting effect was 90% or more, and thus, the aerial part of a cabbage seedling could be protected from damage by a common cutworm.

### Test Example 3

The present compounds (1), (5) and (6) (each 5 mg) were dissolved in a mixture (0.1 ml) of Solgen TW-20 (manufactured by Dai-Ichi Kogyo Seiyaku Co., Ltd.) and acetone (mixing volume ratio; Solgen TW-20:acetone=1:19), and then diluted with ion-exchanged water (3 ml) to prepare a test water dilution of each test compound.

A cabbage seedling was raised until a 2.5-leaf stage in a plug cell tray (each cell volume: 27 ml, depth: 5.0 cm). The test water dilution (3 ml) was drenched on the soil surface at the foot of the cabbage seedling. Five days after the drenching treatment, the root part of the seedling was removed and the stem and leaf part was placed in a polyethylene cup (volume: 180 ml). Into the cup, 10 second-instar larvae of a diamondback moth (Plutella xylostella) were released. After the cup was stored in the chamber at 25° C. for 7 days, the extent of feeding damage of the cabbage seedling by diamondback moth larvae was examined. The damage extent of the cabbage seedling grown in soil which was treated with a test compound (treated section) was compared with the damage extent of the cabbage seedling grown in soil which was not treated with a test compound (non-treated section), and a damage-inhibiting effect was calculated by the aforementioned equation.

As a result, in a section treated with the present compound (1), a section treated with the present compound (5) and a section treated with the present compound (6), the damage-inhibiting effect was 90% or more, and thus, the aerial part of a cabbage seedling could be protected from damage by a diamondback moth.

### Test Example 4

The present compounds (3), (8) and (9) (each 5 mg) were dissolved in a mixture (0.1 ml) of Solgen TW-20 (manufactured by Dai-Ichi Kogyo Seiyaku Co., Ltd.) and acetone (mixing volume ratio; Solgen TW-20:acetone=1:19), and then diluted with ion-exchanged water (3 ml) to prepare a test water dilution of each test compound.

A cabbage seedling was raised until a 2.5-leaf stage in a plug cell tray (each cell volume: 27 ml, depth: 5.0 cm). The test water dilution (3 ml) was drenched on the soil surface at the foot of the cabbage seedling. Five days after the drenching treatment, the root part of the seedling was removed and the stem and leaf part was placed in a polyethylene cup (volume: 180 ml). Into the cup, 10 first-instar larvae of a common cutworm (Spodoptera litura) were released. After the cup was stored in the chamber at 25° C. for 7 days, the extent of feeding damage of the cabbage seedling by common cutworm larvae was examined. The damage extent of the cabbage seedling grown in soil which was treated with a test compound (treated section) was compared with the damage extent of the cabbage seedling grown in soil which was not treated with a test compound (non-treated section), and a damage-inhibiting effect was calculated by the aforementioned equation.

As a result, in a section treated with the present compound (3), a section treated with the present compound (8) and a section treated with the present compound (9), the damage-inhibiting effect was 90% or more, and thus, the aerial part of a cabbage seedling could be protected from damage by a common cutworm.

### Test Example 5

The present compounds (1), (5) and (6) (each 5 mg) were dissolved in a mixture (0.1 ml) of Solgen TW-20 (manufactured by Dai-Ichi Kogyo Seiyaku Co., Ltd.) and acetone (mixing volume ratio; Solgen TW-20:acetone=1:19), and then diluted with ion-exchanged water (5 ml) to prepare a test water dilution of each test compound.

A cabbage seedling was raised until a 2.5-leaf stage in a plug cell tray (each cell volume: 27 ml, depth: 5.0 cm). When the cabbage seedling was transplanted to a 1/5000 a Wagner pot, the soil surface at the bottom of a planting hole dug for transplantation was drenched with the test water dilution (5 ml) and then the cabbage seedling was planted there. Seven days after the transplantation, the root part of the seedling was removed and the stem and leaf part was placed in a polyethylene cup (volume: 180 ml). Into the cup, 10 second-instar larvae of a diamondback moth (Plutella xylostella) were released. After the cup was stored in the chamber at 25° C. for 7 days, the extent of feeding damage of the cabbage seedling by diamondback moth larvae was examined. The damage extent of the cabbage seedling grown in soil which was treated with a test compound (treated section) was compared with the damage extent of the cabbage seedling grown in soil which was not treated with a test compound (non-treated section), and a damage-inhibiting effect was calculated by the aforementioned equation.

As a result, in a section treated with the present compound (1), a section treated with the present compound (5) and a section treated with the present compound (6), the damage-inhibiting effect was 90% or more, and thus, the aerial part of a cabbage seedling could be protected from damage by a diamondback moth.

### Test Example 6

The present compounds (3), (8) and (9) (each 5 mg) were dissolved in a mixture (0.1 ml) of Solgen TW-20 (manufactured by Dai-Ichi Kogyo Seiyaku Co., Ltd.) and acetone (mixing volume ratio; Solgen TW-20:acetone=1:19), and then diluted with ion-exchanged water (5 ml) to prepare a test water dilution of each test compound.

A cabbage seedling was raised until a 2.5-leaf stage in a plug cell tray (each cell volume: 27 ml, depth: 5.0 cm). When the cabbage seedling was transplanted to a 1/5000 a Wagner pot, the soil surface at the bottom of a planting hole dug for transplantation was drenched with the test water dilution (5 ml) and then the cabbage seedling was planted there. Seven days after the transplantation, the root part of the seedling was removed and the stem and leaf part was placed in a polyethylene cup (volume: 180 ml). Into the cup, 10 first-instar larvae of a common cutworm (Spodoptera litura) were released. After the cup was stored in the chamber at 25° C. for 7 days, the extent of feeding damage of the cabbage seedling by common cutworm larvae was examined. The damage extent of the cabbage seedling grown in soil which was treated with a test compound (treated section) was compared with the damage extent of the cabbage seedling grown in soil which was not treated with a test compound (non-treated section), and a damage-inhibiting effect was calculated by the aforementioned equation.

As a result, in a section treated with the present compound (3), a section treated with the present compound (8) and a section treated with the present compound (9), the damage-inhibiting effect was 90% or more, and thus, the aerial part of a cabbage seedling could be protected from damage by a common cutworm.

### Test Example 7

The present compounds (1), (5) and (6) (each 5 mg) were dissolved in a mixture (0.1 ml) of Solgen TW-20 (manufactured by Dai-Ichi Kogyo Seiyaku Co., Ltd.) and acetone (mixing volume ratio; Solgen TW-20:acetone=1:19), and then diluted with ion-exchanged water (10 ml) to prepare a test water dilution of each test compound.

A cabbage seedling was raised until a 6-leaf stage in a polyethylene cup (volume: 980 ml). The test water dilution (10 ml) was drenched on the soil surface at the foot of the cabbage seedling. Seven days after the drenching treatment, the root part of the seedling was removed and the stem and leaf part was placed in a polyethylene cup (volume: 180 ml). Into the cup, 10 second-instar larvae of a diamondback moth (Plutella xylostella) were released. After the cup was stored in the chamber at 25° C. for 7 days, the extent of feeding damage of the cabbage seedling by diamondback moth larvae was examined. The damage extent of the cabbage seedling grown in soil which was treated with a test compound (treated section) was compared with the damage extent of the cabbage seedling grown in soil which was not treated with a test compound (non-treated section), and a damage-inhibiting effect was calculated by the aforementioned equation.

As a result, in a section treated with the present compound (1), a section treated with the present compound (5) and a section treated with the present compound (6), the damage-inhibiting effect was 90% or more, and thus, the aerial part of a cabbage seedling could be protected from damage by a diamondback moth.

### Test Example 8

The present compounds (3), (8) and (9) (each 5 mg) were dissolved in a mixture (0.1 ml) of Solgen TW-20 (manufactured by Dai-Ichi Kogyo Seiyaku Co., Ltd.) and acetone (mixing volume ratio; Solgen TW-20:acetone=1:19), and then diluted with ion-exchanged water (10 ml) to prepare a test water dilution of each test compound.

A cabbage seedling was raised until a 6-leaf stage, in a polyethylene cup (volume: 980 ml). The test water dilution (10 ml) was drenched on the soil surface at the foot of the cabbage seedling. Seven days after the drenching treatment, the root part of the seedling was removed and the stem and leaf part was placed in a polyethylene cup (volume: 180 ml). Into the cup, 10 first-instar larvae of a common cutworm (Spodoptera litura) were released. After the cup was stored in the chamber at 25° C. for 7 days, the extent of feeding damage of the cabbage seedling by common cutworm larvae was examined. The damage extent of the cabbage seedling grown in soil which was treated with a test compound (treated section) was compared with the damage extent of the cabbage seedling grown in soil which was not treated with a test compound (non-treated section), and a damage-inhibiting effect was calculated by the aforementioned equation.

As a result, in a section treated with the present compound (3), a section treated with the present compound (8) and a section treated with the present compound (9), the damage-inhibiting effect was 90% or more, and thus, the aerial part of a cabbage seedling could be protected from damage by a common cutworm.

### INDUSTRIAL APPLICABILITY

According to the present invention, the aerial part of a plant can be protected from damage by a pest by applying a composition containing the present compound to soil where the plant is grown.

1. Use of an (N′-methyl)benzoylurea compound represented by the formula (I): 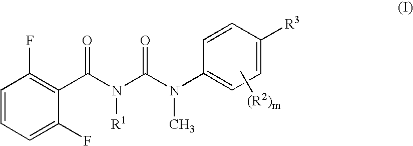
wherein R¹ represents a hydrogen atom, a C1-C6 alkyl group optionally substituted with a halogen atom, a C2-C6 alkenyl group optionally substituted with a halogen atom, a C2-C6 alkynyl group, a C6-C14 aryl group, a C7-C11 aralkyl group, a C2-C6 alkoxyalkyl group, a C7-C14 aryloxyalkyl group, a C3-C6 dialkylaminoalkyl group, a C2-C6 alkylthioalkyl group, a C2-C6 alkylsulfinylalkyl group, a C2-C6 alkylsulfonylalkyl group, a C3-C9 alkoxyalkoxyalkyl group, a C2-C6 alkoxycarbonyl group, a C8-C12 aralkyloxycarbonyl group, a C3-C13 dialkylcarbamoyl group, a C2-C6 alkylcarbonyl group optionally substituted with a halogen atom, a formyl group, a C1-C5 alkylsulfonyl group optionally substituted with a halogen atom, or a C6-C10 arylsulfonyl group,
R² represents a halogen atom, or a C1-C4 alkyl group optionally substituted with a halogen atom, and
R³ represents a halogen atom, a C1-C4 alkyl group optionally substituted with a halogen atom, a C1-C4 alkoxy group optionally substituted with a halogen atom, a C2-C6 alkoxyalkoxy group optionally substituted with a halogen atom, a C2-C4 alkenyloxy group optionally substituted with a halogen atom, or a C2-C4 alkynyloxy group optionally substituted with a halogen atom, and
m represents an integer of 0 to 4, in soil treatment for protecting the aerial part of a plant from damage by a pest.

2. The use according to claim 1, wherein the pest is a lepidopteran or a thrips.

3. A composition for soil treatment for protecting the aerial part of a plant from damage by a pest, which comprises an (N′-methyl)benzoylurea compound represented by the formula (I): 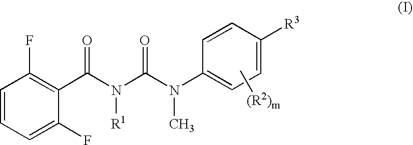
wherein R¹ represents a hydrogen atom, a C1-C6 alkyl group optionally substituted with a halogen atom, a C2-C6 alkenyl group optionally substituted with a halogen atom, a C2-C6 alkynyl group, a C6-C14 aryl group, a C7-C11 aralkyl group, a C2-C6 alkoxyalkyl group, a C7-C14 aryloxyalkyl group, a C3-C6 dialkylaminoalkyl group, a C2-C6 alkylthioalkyl group, a C2-C6 alkylsulfinylalkyl group, a C2-C6 alkylsulfonylalkyl group, a C3-C9 alkoxyalkoxyalkyl group, a C2-C6 alkoxycarbonyl group, a C8-C12 aralkyloxycarbonyl group, a C3-C13 dialkylcarbamoyl group, a C2-C6 alkylcarbonyl group optionally substituted with a halogen atom, a formyl group, a C1-C5 alkylsulfonyl group optionally substituted with a halogen atom, or a C6-C10 arylsulfonyl group,
R² represents a halogen atom, or a C1-C4 alkyl group optionally substituted with a halogen atom, and
R³ represents a halogen atom, a C1-C4 alkyl group optionally substituted with a halogen atom, a C1-C4 alkoxy group optionally substituted with a halogen atom, a C2-C6 alkoxyalkoxy group optionally substituted with a halogen atom, a C2-C4 alkenyloxy group optionally substituted with a halogen atom, or a C2-C4 alkynyloxy group optionally substituted with a halogen atom, and
m represents an integer of 0 to 4.

4. A method for protecting the aerial part of a plant from damage by a pest, which comprises a step of applying the composition according to claim 3 to soil where the plant is grown.

5. The method according to claim 4, wherein the plant is a seedling.

6. The method according to claim 4, wherein the pest is a lepidopteran or a thrips.

7. The method according to claim 4, wherein application of the composition is carried out by soil drenching.

8. The method according to claim 5, wherein the pest is a lepidopteran or a thrips.

9. The method according to claim 5, wherein application of the composition is carried out by soil drenching.
